(19) United States
(12) Patent Application Publication
(10) Pub. No.: US 2018/0125414 A1
(43) Pub. Date: May 10, 2018

(54) PATIENT SUPPORT APPARATUSES WITH MOBILITY ASSESSMENT

(71) Applicant: Stryker Corporation, Kalamazoo, MI (US)

(72) Inventors: Patrick Lafleche, Kalamazoo, MI (US); Marko N. Kostic, Portage, MI (US); Sujay Sukumaran, Portage, MI (US); Jonathan Mark Greenbank, Plainwell, MI (US); Madhu Thomas, London (CA); Manikantan Seshadrinathan, Gurgaon (IN)

(21) Appl. No.: 15/809,351

(22) Filed: Nov. 10, 2017

Related U.S. Application Data
(60) Provisional application No. 62/420,264, filed on Nov. 10, 2016.

(51) Int. Cl. A61B 5/00; A61B 5/01; A61B 5/11; A61F 5/30; A61G 7/057; A61G 1/04; G06F 19/00

(52) U.S. Cl. CPC A61B 5/447; G06Q 10/109; A61B 5/0077; A61B 5/01; A61B 5/1118; A61B 5/1116; A61F 5/30; A61G 7/05769; A61G 1/04; G06F 19/3431; A61B 2562/0252; A61B 2562/0219; A61B 2562/0247; A61G 2203/10; A61G 2203/44; A61G 2203/70; A61B 5/6892

(57) ABSTRACT

A patient support apparatus is provided, such as a bed, cot, stretcher, or the like, that includes a sensor adapted to detect movement of the occupant while the occupant is supported on the support apparatus. A controller monitors outputs from the sensor in response to a mobility assessment control being activated. After monitoring the outputs for a period of time, the controller generates a mobility score based on the outputs from the sensor. In some embodiments, the controller use outputs from the sensor to determine which region of a defined surface area the patient has moved to. The controller records these visited regions over a time period and uses them to generate the mobility assessment. The mobility assessment provides an objective measure of an important factor used in predicting a patient's risk of developing pressure ulcers.

### CROSS-REFERENCE TO RELATED APPLICATIONS

This application claims priority to U.S. provisional patent application Ser. No. 62/420,264 filed Nov. 10, 2016, by inventors Patrick Lafleche et al. and entitled PATIENT SUPPORT APPARATUSES WITH MOBILITY ASSESSMENT, the complete disclosure of which is incorporated herein by reference.

### BACKGROUND

The present disclosure relates to patient support apparatuses, such as beds, cots, stretchers, operating tables, recliners, or the like. More specifically, the present disclosure relates to patient support apparatuses that are adapted to reduce incidences of pressure ulcers.

Some existing hospital beds and/or stretchers include a reminder feature that prompts a caregiver to turn a patient of the hospital bed and/or stretcher at certain intervals. The turning of the patient is intended to relieve any areas of the patient's body that are experiencing high pressure so that the likelihood of a pressure ulcer developing in those areas is reduced. The decision to use the turning reminder feature of the bed/stretcher may be based upon a conventional pressure ulcer risk score, such as the Braden scale, the Norton scale, or some other score associated with a particular patient. Such scores, however, are often not as accurate as is desirable, leading to either overinclusion of patients who don't need to be turned, or underinclusion of patients who should be turned. The former can lead to unnecessary expenditures of labor while the latter can lead to patients developing otherwise preventable pressure ulcers.

### SUMMARY

According to various embodiments, the present disclosure provides a patient support apparatus that is adapted to provide improved data for preventing the development of pressure ulcers in occupants of the patient support apparatus. The improved data is generated from one or more objective tests that are more accurate indicators of the susceptibility of a particular individual to developing pressure ulcers. The objective tests require very little labor on the part of caregivers or other personnel. The objective tests involve monitoring movement of the occupant over a time period and analyzing the kinds and/or amounts of movement of the occupant over the time period in order to determine an assessment of the mobility of the occupant. The mobility score provides a useful tool for assessing the risk of pressure ulcers developing and therefore enables caregivers assigned to the occupant to take appropriate steps to reduce the likelihood of pressure ulcer development.

According to one embodiment, a patient support apparatus is provided that includes a frame, a support surface, a sensor, a user interface, and a controller. The support surface is adapted to support thereon an occupant of the patient support apparatus. The sensor detects movement of the occupant while the occupant is supported on the support surface. The user interface includes a mobility assessment control, and the controller monitors outputs from the sensor in response to the mobility assessment control being activated. The controller also generates a mobility score based on the outputs from the sensor over a predetermined time period.

According to other aspects, the controller displays the mobility score on the user interface.

The sensor may include one or more of a video camera, a thermal image sensor, an ultrasonic sensor, an array of pressure sensors, and a plurality of force sensors adapted to detect downward forces exerted by the occupant on the support surface.

In some embodiments, the sensor comprises a plurality of load cells and the controller is adapted to use the outputs from the load cells to determine a distribution of the occupant's weight on the support surface and to detect changes in the distribution of the occupant's weight. The controller may further record how often changes in the distribution of the occupant's weight occurs over the predetermined time period and/or magnitudes of the changes in the distribution of the occupant's weight. The controller is adapted to use the records of the frequency and/or magnitudes of the weight distribution changes when generating the mobility score.

A transceiver is included in some embodiments that is used by the controller to transmit the mobility score to one or more devices positioned off-board of the patient support apparatus, such as, but not limited to, a pager, phone, nurse's station, smart phone, laptop computer, etc. In some embodiments, the controller uses the transceiver to automatically forward the mobility score to a caregiver communication device after the mobility score has been determined, thereby removing any need for the caregiver to be near to the patient support apparatus when the assessment is completed.

In some embodiments, the controller is further adapted to recommend an action based on a value of the mobility score wherein the action is designed to reduce a possibility of the occupant developing pressure ulcers while supported on the support surface. The action may comprise recommending one or more of the following: (1) using a particular mattress on the support surface of the patient support apparatus; (2) turning the occupant at a particular time interval while on the support surface of the patient support apparatus; (3) inflating a mattress on the support surface to a particular level; and (4) using a pressure reducing device with the occupant while on the support surface.

In still other embodiments, the controller is further adapted to define an area over which the occupant may move while supported on the support surface; divide the area into a plurality of regions; determine which region the occupant's location on the support surface corresponds to; and generate a record of the regions which the occupant's location has corresponded to during the predetermined time period.

The controller analyzes the record of the regions in order to determine how many times the occupant's location has visited each region during the predetermined time period. The controller may determine one or more of the following: (1) a concentration value of the visited regions; (2) a distribution value of the visited regions; (3) how many times the occupant has moved more than a threshold number of regions; and (4) how many times the occupant has moved to the same region.

In some embodiments, the controller automatically monitors the outputs from the sensor without activation of the mobility assessment control. In such embodiments, the controller may automatically monitor the outputs from the sensor and generate the mobility score in response to at least one of the following: a changing of a work shift for a nurse assigned to the occupant; a passage of a predetermined amount of time; an assignment of a new occupant to the patient support apparatus; and an occurrence of a predefined event.

According to another embodiment, a patient support apparatus is provided that includes a frame, a support surface, a sensor, and a controller. The support surface is adapted to support thereon an occupant of the patient support apparatus. The sensor monitors movement of the occupant while the occupant is supported on the support surface. The movement is monitored by the sensor over an area that is subdivided into regions. The controller uses outputs from the sensor to determine which region of the area the occupant's location on the support surface corresponds to. The controller further generates a record of the regions which the occupant's location has corresponded to over a time period.

According to other aspects, the controller analyzes the record to determine how many times the occupant has visited each region over the time period. The controller may also determine one or more of the following: (1) a concentration value of the visited regions; (2) a distribution value of the visited regions; (3) how many times the occupant has moved more than a threshold number of regions; (4) how many times the occupant has moved to the same region; (5) one or more clusters of the visited regions and to display the clusters; and (6) a mobility score for the occupant based on the outputs from the sensor taken over the time period.

In some embodiments, the controller determines if the occupant exits the patient support apparatus during the time period and to use data regarding the occupant's exit when generating the mobility score.

According to still another embodiment, a method for assessing the mobility of an occupant of a patient support apparatus is provided. The method includes gathering a collection of sensor readings taken over a time period while the occupant of the patient support apparatus is supported on a support surface of the patient support apparatus; determining a total amount of movement of the occupant over the time period from the collection of sensor readings; and generating a mobility score based on the total amount of movement.

The step of determining a total amount of movement of the occupant over the time period comprises, in some embodiments, collecting outputs from a plurality of force sensors adapted to detect downward forces exerted by the occupant while supported on the support surface; and determining changes in a distribution of the downward forces exerted by the occupant over the time period.

In other embodiments, the step of determining a total amount of movement of the occupant over the time period comprises at least one of the following: (1) collecting images of the occupant during the time period and analyzing the images to determine the total amount of movement of the occupant; and (2) collecting pressure readings of the occupant during the time period and analyzing the pressure readings to determine the total amount of movement of the occupant.

The area over which the occupant may move is defined in a plane parallel to, or coincident with, a plane defined by at least a portion of the support surface.

In some embodiments, the method further includes performing a k-means clustering analysis of the record of the visited regions to determine at least one cluster of the visited regions.

Before the various embodiments disclosed herein are explained in detail, it is to be understood that the claims are not to be limited to the details of operation or to the details of construction and the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus according to one embodiment of the disclosure;

FIG. 2 is a perspective view of a litter frame of the patient support apparatus of FIG. 1;

FIG. 3 is a perspective view of a base of the patient support apparatus of FIG. 1;

FIG. 4 is a block diagram of a control system for a patient support apparatus, such as the patient support apparatus of FIG. 1;

FIG. 5 is a diagram of an illustrative record of regions visited by an occupant of the patient support apparatus that, in some embodiments, is generated and analyzed by the control system; and

FIG. 6 is an example of an arbitrary mobility assessment score message displayed on a display of the patient support apparatus of FIG. 1.

### DETAILED DESCRIPTION OF THE EMBODIMENTS

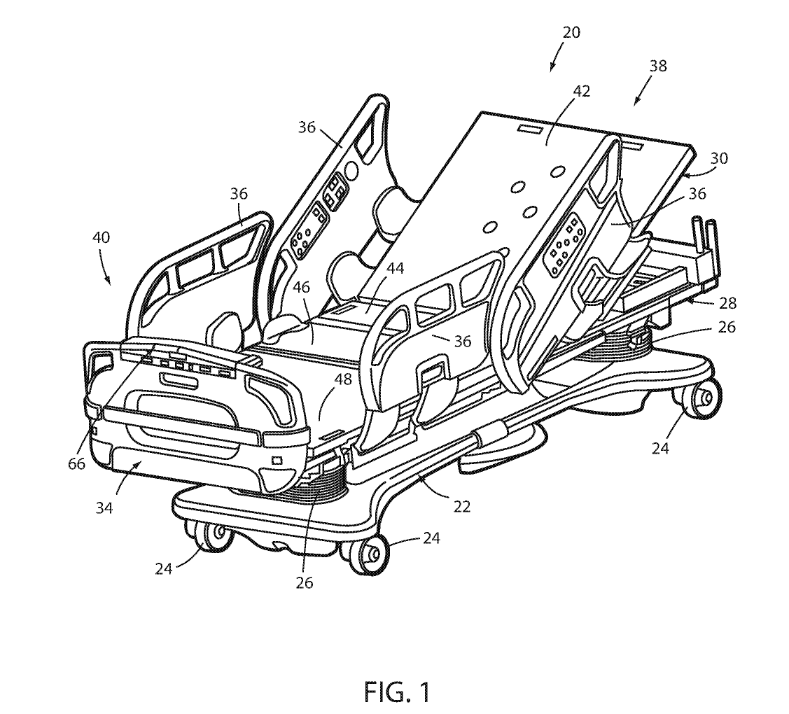

An illustrative patient support apparatus 20 according to a first embodiment is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a gurney, a recliner, an operating table, a residential bed, or any other structure capable of supporting a patient, whether stationary or mobile and/or whether medical or residential.

In general, patient support apparatus 20 includes a base 22 having a plurality of wheels 24, a pair of lifts 26 supported on the base, a litter frame 28 supported on the lifts 26, and a support deck 30 supported on the litter frame 28. Patient support apparatus 20 further includes a footboard 34 and a plurality of siderails 36. Siderails 36 are all shown in a raised position in FIG. 1 but are each individually movable to a lowered position in which ingress into, and egress out of, patient support apparatus 20 is not obstructed by the lowered siderails 36.

Lifts 26 are adapted to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted. That is, litter frame 28 includes a head end 38 and a foot end 40, each of whose height can be independently adjusted by the nearest lift 26. Patient support apparatus 20 is designed so that when an occupant lies thereon, his or her head will be positioned adjacent head end 38 and his or her feet will be positioned adjacent foot end 40.

Litter frame 28 provides a structure for supporting support deck 30, footboard 34, and siderails 36. Support deck 30 provides a support surface for a mattress (not shown in FIG. 1), or other soft cushion, so that a person may lie and/or sit thereon. The top surface of the mattress or other cushion forms a support surface for the occupant. Support deck 30 is made of a plurality of sections, some of which are pivotable about generally horizontal pivot axes. In the embodiment shown in FIG. 1, support deck 30 includes a head section 42, a seat section 44, a thigh section 46, and a foot section 48. Head section 42, which is also sometimes referred to as a Fowler section, is pivotable about a generally horizontal pivot axis between a generally horizontal orientation (not shown in FIG. 1) and a plurality of raised positions (one of which is shown in FIG. 1). Thigh section 46 and foot section 48 may also be pivotable about generally horizontal pivot axes.

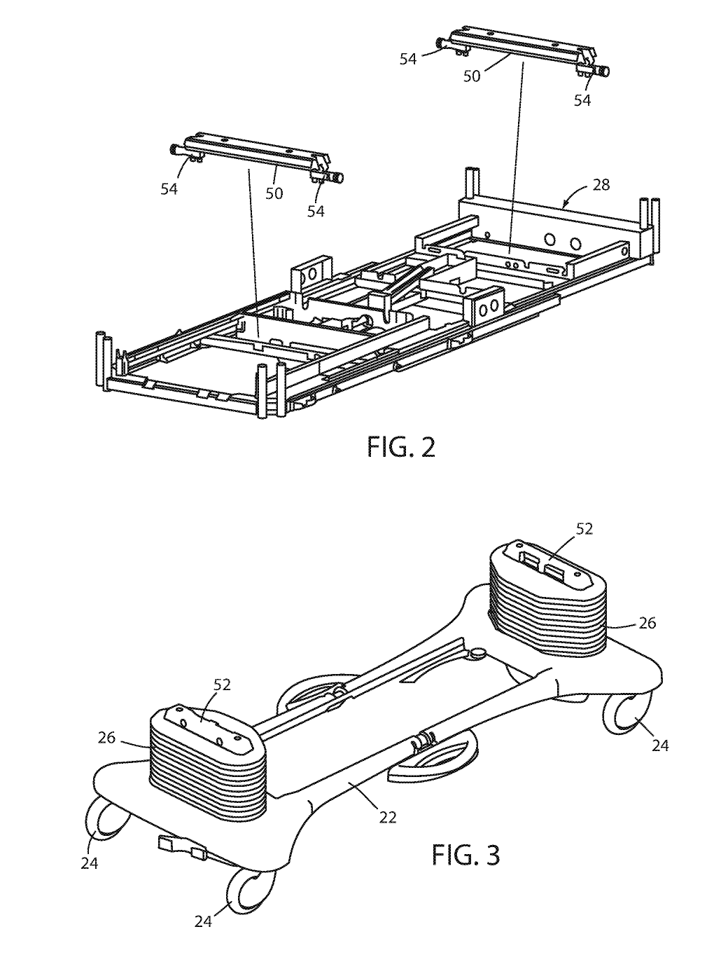

FIG. 2 illustrates in greater detail litter frame 28 separated from lifts 26 and base 22. Litter frame 28 is also shown in FIG. 2 with support deck 30 removed. Litter frame 28 is supported by two lift header assemblies 50. A first one of the lift header assemblies 50 is coupled to a top 52 (FIG. 3) of a first one of the lifts 26, and a second one of the lift header assemblies 50 is coupled to the top 52 of the second one of the lifts 26. Each lift header assembly 50 includes a pair of load cells 54. Load cells 54 may be replaced by other force sensors, including, but not limited to, linear variable displacement transducers and/or any one or more capacitive, inductive, and/or resistive transducers that are configured to produce a changing output in response to changes in the force exerted against them.

Although the illustrated embodiment of patient support apparatus 20 includes a total of four load cells 54 (FIG. 3), it will be understood by those skilled in the art that different numbers of load cells 54 may be used in accordance with the principles of the present disclosure. Load cells 54 are configured to support litter frame 28. More specifically, load cells 54 are configured such that they provide complete and exclusive mechanical support for litter frame 28 and all of the components that are supported on litter frame 28 (e.g. support deck 30, footboard 34, the headboard, siderails 36, etc.). Because of this construction, load cells 54 are adapted to detect the weight of not only those components of patient support apparatus 20 that are supported by litter frame 28 (including litter frame 28 itself), but also any objects or persons who are wholly or partially being supported by support deck 30. The outputs of load cells 54 may be part of a control system described in greater detail below.

Load cells 54 are adapted to detect downward forces exerted by an occupant of support deck 30. Thus, when an occupant is positioned on support deck 30 and substantially still (i.e. not moving in a manner in which patient acceleration forces are exerted against support deck 30), load cells 54 detect the weight of the occupant (as well as the weight of any components of patient support apparatus 20 that are supported—directly or indirectly—by load cells 54). In some embodiments discussed more below, load cells 54 are also or alternatively used to determine a center of gravity of the occupant. In alternative embodiments, the outputs from load cells 54 are analyzed, not to determine a center of gravity, but instead to determine a weight distribution and/or a change in weight distribution, such as by determining one or more ratios of the relative weights sensed by the load cells 54. Other types of sensors may also or alternatively be used for determining the occupant's weight and/or movement instead of load cells.

The mechanical construction of patient support apparatus 20, as shown in FIGS. 1 and 2, is the same as, or nearly the same as, the mechanical construction of the Model 3002 S3 bed manufactured and sold by Stryker Corporation of Kalamazoo, Mich. This mechanical construction is described in greater detail in the Stryker Maintenance Manual for the MedSurg Bed, Model 3002 S3, published in 2010 by Stryker Corporation of Kalamazoo, Mich., the complete disclosure of which is incorporated herein by reference. It will be understood by those skilled in the art that patient support apparatus 20 can be designed with other types of mechanical constructions, such as, but not limited to, those described in commonly assigned, U.S. Pat. No. 7,690,059 issued to Lemire et al., and entitled HOSPITAL BED; and/or commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The mechanical construction of patient support apparatus 20 may also take on forms different from what is disclosed in the aforementioned references.

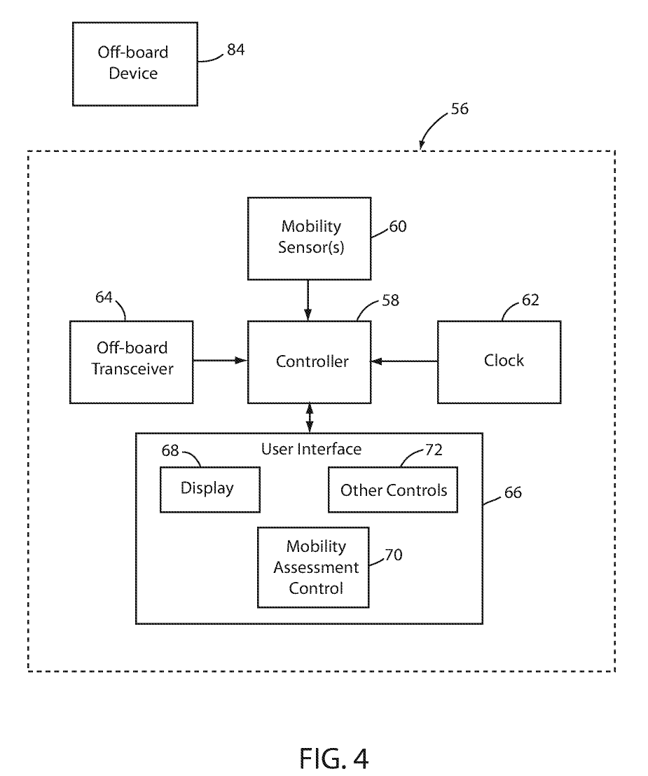

FIG. 4 illustrates a control system 56 that is usable on patient support apparatus 20. Control system 56 includes a controller 58, one or more mobility sensors 60, a clock 62, an off-board transceiver 64, and a user interface 66. User interface 66 includes, in the illustrated embodiment, a display 68, a mobility assessment control 70, and one or more other controls 72. As will be discussed in greater detail below, the one or more mobility sensors 60 are adapted to detect movement of the patient while the patient is supported on patient support apparatus 20. The outputs from the one or more mobility sensors 60 are fed to controller 58 which analyzes them and calculates a mobility score for the patient. The mobility score represent an objective assessment of the degree of the patient's mobility.

The mobility score is displayed on display 68 and, in some cases, transmitted via off-board transceiver 64 to one or more off-board devices 84, such as, but not limited to, a nurse's cell phone, pager, badge, personal digital assistant, nurse's station, or other electronic communication device that is adapted to share the mobility score with the caregivers associated with the patient. Once the mobility score is known, the caregivers can use this to determine what steps, if any, should be taken to mitigate the risk of pressure ulcers developing on that particular patient. In some situations, the caregiver may use the object mobility score as a factor that is input into another risk assessment took, such as the Braden scale. In other situations, the caregiver may use the mobility score as a stand-alone assessment of pressure ulcer risk. Regardless of the specific manner in which the score is used by the caregiver, the mobility score represents an objective measurement of mobility, which is an important factor in determining the risk of pressure ulcers. The mobility score generated by controller 58 therefore allows caregivers to more accurately target their risk mitigation efforts toward patients needing assistance and to avoid expending time and resources efforts where such efforts are likely not necessary.

The manner in which controller 58 generates a mobility score can be better understood by describing in more detail the structure and operation of the individual components of control system 56, which will now be done, starting with controller 58. Controller 58 is a microcontroller in at least one embodiment. It will be understood, however, that controller 58 may take on other forms. In general, controller 58 may include any one or more microprocessors, microcontrollers, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. The instructions followed by controller 58 in carrying out the functions described herein, as well as the data necessary for carrying out these functions, are stored in memory (not labeled) accessible to controller 58.

Mobility sensors 60 may take on a wide variety of forms. In the embodiment of patient support apparatus 20 shown in FIGS. 1-3, mobility sensors 60 correspond to load cells 54. In this embodiment, the outputs from the load cells 54 are continuously, or repeatedly, monitored to determine the location and/or amount of movement of the patient while supported on support deck 30. In one such embodiment, controller 58 calculates the center of gravity of the occupant using the outputs from the four load cells and monitors and records the movement of this center of gravity over time. This calculation of the center of gravity may be carried out in the manner disclosed in commonly assigned U.S. Pat. No. 5,276,432 issued to Travis and entitled PATIENT EXIT DETECTION MECHANISM FOR HOSPITAL BED, the complete disclosure of which is incorporated herein by reference. Alternatively, or additionally, the center of gravity of the patient may be determined using the techniques disclosed in commonly assigned U.S. patent application Ser. No. 14/918,003 filed Oct. 20, 2015, by inventors Marko Kostic et al. and entitled EXIT DETECTION SYSTEM WITH COMPENSATION.

In alternative embodiments, mobility sensors 60 may include any of the sensors used to determine an occupant's location with respect to patient support apparatus 20 that are disclosed in any of the following commonly assigned U.S. patent application Ser. No. 14/003,157 filed Mar. 2, 2012, by inventors Joshua Mix et al. and entitled SENSING SYSTEM FOR PATIENT SUPPORTS; Ser. No. 14/928,513 filed Oct. 30, 2015, by inventors Richard Derenne et al. and entitled PERSON SUPPORT APPARATUSES WITH PATIENT MOBILITY MONITORING; Ser. No. 14/873,734 filed Oct. 2, 2015, by inventors Marko Kostic et al. and entitled PERSON SUPPORT APPARATUSES WITH MOTION MONITORING; Ser. No. 14/692,871 filed Apr. 22, 2015, by inventors Marko Kostic et al. and entitled PERSON SUPPORT APPARATUS WITH POSITION MONITORING; Ser. No. 14/578,630 filed Dec. 22, 2014, by inventors Richard Derenne et al. and entitled VIDEO MONITORING SYSTEM; and 62/253,167 filed Nov. 20, 2015, by inventors Marko Kostic et al. and entitled PERSON SUPPORT APPARATUSES WITH ACCELERATION DETECTION, the complete disclosures of all of which are incorporated herein by reference.

Clock 62 is any suitable timekeeping device able to keep track of an elapsed amount of time after a user activates mobility assessment control 70, as will be discussed in greater detail below. In some embodiments, clock 62 is operated in accordance with the principles disclosed in commonly assigned U.S. patent application Ser. No. 62/361,092 filed Jul. 12, 2016, by inventors Anuj Sidhu et al. and entitled PATIENT SUPPORT APPARATUSES WITH CLOCKS, the complete disclosure of which is incorporated herein by reference. Other types of clocks may be used, including crystal oscillators and/or other timing devices commonly incorporated into microprocessors and/or microcontrollers. In other words, clock 62 may be integrated into controller 58 in some embodiments.

Off-board transceiver 64 allows control system 56 to communicate with one or more off-board devices 84. Transceiver 64 may be coupled to a port for receiving one or more wires or cables, or it may be a wireless transceiver coupled to an antenna, or other wireless transmitting means (e.g. infrared transmitter). In some embodiments, one or more transceivers 64 are included that communicate both via wire and wirelessly. When a port is included, the port may be an Ethernet port (e.g. an RJ-45 port) for coupling to an Ethernet cable (e.g. Cat5, Cat6, etc.) that is coupled to a healthcare facility computer network. Alternative, a Universal Serial Bus (USB) port, or other type of port may be included that is used to communicate with one or more other types of off-board devices 84 and/or with a healthcare facility computer network. If transceiver 64 is a wireless transceiver, it may be a WiFi transceiver (IEEE 802.11) that communicates with one or more wireless access points of the healthcare facility network. Alternatively, transceiver 64 may be implemented as a wireless ZigBee transceiver (IEEE 802.15.), a wireless Bluetooth transceiver (IEEE 802.15.1), and/or another type of transceiver.

User interface 66 communicates with controller 58 and enables a user of patient support apparatus 20 to control one or more aspects of patient support apparatus 20. User interface 66 is implemented in the embodiment shown in FIG. 1 as a control panel having a lid (flipped down in FIG. 1) underneath which is positioned a plurality of controls, including mobility assessment control 70 and one or more additional controls 72. These controls (70 and 72)—which may be buttons, dials, switches, or other devices—allows a user to control various aspects of control system 56, such as, but not limited to, controlling an exit detection system, moving the various components of patient support apparatus 20, taking weight readings from an onboard scale system (if present), controlling communications with off-board device 84 (which may, as noted, comprise a healthcare facility computer network), activating and deactivating the mobility assessment carried out in response to the manipulation of mobility assessment control 70, and changing configuration settings associated with patient support apparatus 20 (including, but not limited to, configurations associated with the mobility assessment carried out in response to activation of mobility assessment control 70).

Although FIG. 1 illustrates user interface 66 mounted to footboard 34, it will be understood that user interface 66 can be positioned elsewhere, and/or that one or more additional user interfaces can be added to patient support apparatus 20 in different locations, such as the siderails 36, for controlling various aspects of patient support apparatus 20. In addition, one or more user interfaces may be communicatively coupled to patient support apparatus 20 but physically positioned remote from patient support apparatus 20, such as, but not limited to, a computer tablet, a smart phone, a computer station, etc.

User interface 66 also includes a display 68, which may be a Liquid Crystal Display, or any other type of display on which graphics and/or text is able to be displayed to the user.

As noted previously, control system 56 is adapted to automatically carry out an objective assessment of the mobility of an occupant of patient support apparatus 20 in response to a user activating mobility assessment control 70. This enables the caregiver associated with the occupant to determine what level of risk the patient occupying patient support apparatus 20 is at for the development of pressure ulcers, and to thereby take the appropriate counter measures for preventing these from developing (e.g. turning the patient, using a special mattress with the patient, using a heel care boot, etc.).

A caregiver begins the mobility assessment after the patient has entered patient support apparatus 20 by pressing a button, icon, or the like that corresponds to mobility assessment control 70 (or taking another action that activates mobility assessment control 70 if it is not press-activated). Once the mobility assessment control 70 has been activated, controller 58 begins monitoring the outputs from mobility sensors 60 and continues to do so for a predetermined period of time. In some embodiments, the predetermined period of time may be on the order of fifteen to thirty minutes. However, it will be understood by those skilled in the art that other time periods may be used. During this predetermined time period, controller 58 records and/or analyzes the outputs from mobility sensors 60 and determines how much the patient has moved during the time period.

In some embodiments, control system 56 is configured to automatically start the mobility assessment based on one or more triggers. Such triggers include, but are not limited to, any one or more of the following: a changing of a work shift for a caregiver assigned to the occupant; a passage of a predetermined amount of time; an assignment of a new occupant to the patient support apparatus; the return of the patient to patient support apparatus 20, and an occurrence of a predefined event.

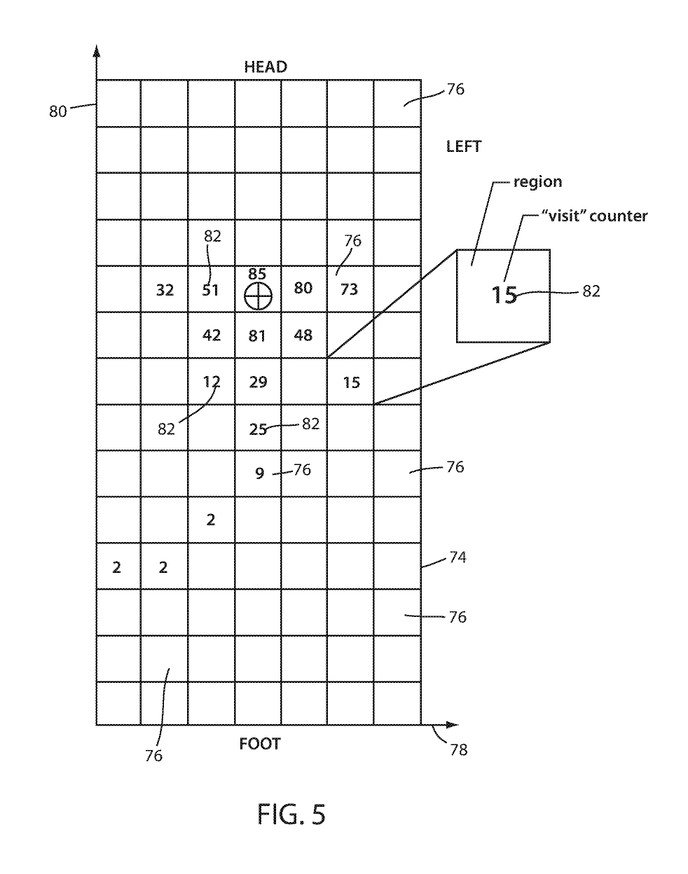

Determining the amount of movement of the patient during the time period of the mobility assessment can be carried out in a number of manners. One illustrative manner is shown in FIG. 5. Other manners may also be used. FIG. 5 shows a planar area 74 that is divided into a plurality of regions 76. The specific number of regions 76 shown in FIG. 5 is merely one example of the number of regions that may be used, and both larger and smaller numbers of regions 76 may be used in different embodiments. Still further, although regions 76 are depicted as uniformly sized, this need not be the case. Indeed, in some embodiments, the regions 76 may be dynamically defined and have sizes and/or shapes that change during movement of the patient.

Planar area 74 corresponds generally to the plane of support deck 30 when sections 42-48 are in their horizontal orientation. That is, planar area 74 generally represents a plan view of the support surface of patient support apparatus 20 when looking down on patient support apparatus 20. The top of planar area 74 in FIG. 5 thus corresponds to head end 38 of patient support apparatus 20 while the bottom of planar area 74 in FIG. 5 corresponds to foot end 40 of patient support apparatus 20. When control system 56 is configured to compute the center of gravity of the patient while supported on patient support apparatus, planar area 74 may be the same plane in which the center of gravity is calculated. In such embodiments, planar area 74 may be assigned an X-axis 78 and a Y-axis 80, as shown in FIG. 5. However, it will be understood by those skilled in the art that these coordinate axes may be changed, and that any other suitable frame of reference and/or coordinate definitions (e.g. polar coordinates) may be used. Planar area 74 may also be modified to include multiple planes (such as to accommodate the different sections 42-48 of deck 30) and/or to oriented at a non-zero angle with respect to the generally horizontal planar area defined by litter frame 28.

In those embodiments of patient support apparatus 20 where mobility sensors 60 include one or more image sensors (thermal and/or visual), planar area 74 may be adjusted to generally match a plane that encompasses the field of view of the image sensor (i.e. a plane parallel to the plane defined by the sensor element of the image sensor). The plane is divided into a plurality of regions that, in combination, define the image captured by the image sensor. Further, if the image sensors are adapted to detect, either alone or in conjunction with other mobility sensors 60, three dimensional movement of the patient, then planar area 74 may be modified to be a volume having volume elements (e.g. voxels) instead of a plane having planar regions, as would be known to one of ordinary skill in the art.

In the embodiment shown in FIG. 5, control system 56 repetitively computes the center of gravity of the patient during the time period of the mobility assessment (which is triggered by control 70). After each computation of the center of gravity, controller 58 determines which region of planar area 74 the center of gravity falls within. A region 76 that encompasses the calculated center of gravity is referred to as a visited region, and each time the center of gravity falls within a particular region 76, that region 76 is considered to have been visited by the patient. Controller 58 keeps track of how many times each region is visited by the center of gravity as well as, in some embodiments, the time and/or sequence at which each region is visited. In the example shown in FIG. 5, the visit count for each region is shown by identifier 82. Controller 58 continues to determine and record these visit counts throughout the entire time period during which the mobility assessment is occurring.

After the time period has expired, controller 58 analyzes the visit counts to determine how much movement the patient performed during the time period of the mobility assessment. The higher the degree of movement, the less the risk of developing pressure ulcers. The manner in which controller 58 quantifies the amount of movement of the patient over the assessment time period can vary in different embodiments. In one embodiment, controller 58 uses a K-means algorithm to define one or more clusters of the visited regions 76. The K-means clustering provides quantitative values about the concentration and distribution of patient location in particular areas of the patient support apparatus. Additionally, or alternatively, controller 58 may use any other data clustering algorithms, including, but not limited to: the K-means++; K-median; K-medoids; Fuzzy clustering by Local Approximation of MEmbership (FLAME); the Hoshen-Kopelman algorithm; Unweighted Pair Group Method with Arithmetic Mean (UPGMA); and a variety of other known data clustering techniques.

In addition to, or alternatively to, performing one or more data clustering analyses, controller 58 is configured in some embodiments to analyze the visited regions (including sequence and/or timing) to determine specific movements of the patient, such as, but not limited to, sitting up, leaning back, rolling to or from his or her back to his or her side, or vice versa, and others. Still further, controller 58 may be additionally or alternatively configured to compute a total distance traveled by the patient during the mobility assessment time period. Such distance may be measured in regions. For example, if two consecutive center of gravity readings are taken for the same region, the traveled distance is zero. If two consecutive readings are taken that correspond to neighboring regions 76, the traveled distance may be assigned a value of one. For movement from one region 76 to another region 76 that are not neighbors of each other, the distance may be determined in any suitable manner, such as the number of regions between, an algebraic computation of distance, etc.

Controller 58 is also configured in some embodiments to take into account the speed of the movement between regions, the kinetic energy involved in the patient's movement between regions, and/or the weight of the patient. When all else is equal, movement at greater speeds and movement of greater patient weights are considered by controller 58 to be indicative of greater mobility. The direction of the movement may also be considered in some embodiments wherein the lack of side-to-side movement and/or the lack of head-to-foot end movement leads to a lowered score, even if ample movement in another direction is detected. Indeed, in some embodiments, controller 58 determines a mobility score based upon evaluating whether a threshold number of trips (i.e. paths traced by the visited regions) of the patient's center of gravity occurred that were greater than a predefined distance and performed with more than a predefined amount of deviation in the respective directions of the trips.

In still other embodiments, one or more statistical dispersion calculations (e.g. statistical variance, standard deviations, interquartile ranges, etc.) may be made by controller 58 with respect to the visited clusters, either in lieu of or in addition to, the aforementioned clustering analyses.

As will be apparent from the foregoing description, the specific analysis performed by controller 58 of the visited regions may vary widely from embodiment to embodiment. However, regardless of the specific set of algorithm(s) used to process the visited regions, controller 58 is configured to compare the outputs of the analyzed region visit data to one or more predetermined thresholds. The thresholds are used to convert the analysis of the visited regions 76 to a mobility assessment score. The mobility assessment score is then output to caregiver.

In those embodiments of patient support apparatus 20 wherein controller 58 determines the mobility score based upon analyses of the movement of the patient's center of gravity, controller 58 may be additionally programmed to differentiate changes in the center of the gravity caused by movement of the patient from changes in the center of gravity caused by the addition or removal of objects from the patient support apparatus 20. In such situations, controller 58 does not include within its statistical analyses center of gravity movement that is caused by the addition or removal of objects, but instead only includes movement of the center of gravity due to movement of the patient. Suitable techniques that may be used by controller 58 for differentiating these types of movement are disclosed in commonly assigned U.S. patent application Ser. No. 14/873,734 filed Oct. 2, 2015, by inventors Marko Kostic et al. and entitled PERSON SUPPORT APPARATUSES WITH MOTION MONITORING; and commonly assigned Patent Cooperation Treaty application number PCT/US2014/02630 filed Mar. 13, 2014, by inventors Michael Hayes et al. and entitled PATIENT SUPPORT APPARATUS WITH PATIENT INFORMATION SENSORS, the complete disclosures of both of which are incorporated herein by reference.

Controller 58 is also programmed, in some embodiments, to take into account any detected exit of the patient during the time period when the mobility assessment is operating. In some of these embodiments, controller 58 automatically pauses the mobility assessment while the patient is out of patient support apparatus 20 and automatically restarts the assessment when the patient returns. In other embodiments, controller 58 automatically starts the assessment over from scratch when the patient returns and ignores the previously accumulated movement data. In any of these embodiments, the act of exiting without caregiver assistance may be assigned a score that is used in the calculation of the mobility assessment value.

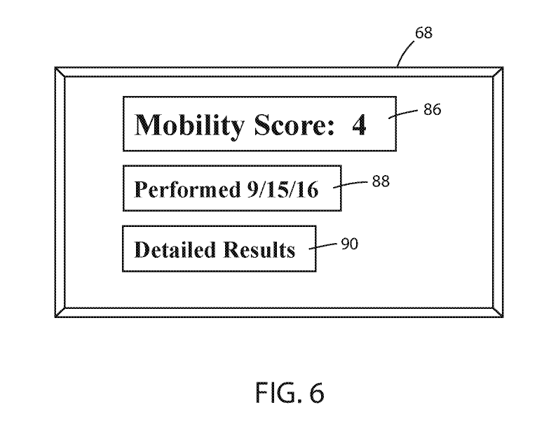

An example of one manner of displaying the mobility assessment score is shown in FIG. 6. As can be seen therein, a mobility score 86 was assigned a value of 4. This score may be based on any suitable scale, such as a scale of 1 to 10, a scale of 1 to 5, or any other suitable numeric scale. Further, the score may be ranked in any order such that, in some embodiments, higher numbers are indicative of greater mobility and in other embodiments lower numbers are indicative of greater mobility. As yet another alternative, the score may be letter-based, or use some other scale that is non-numeric.

The assessment score 86 shown in FIG. 6 is displayed on display 68 of user interface 66. This score 86 is displayed automatically upon the completion of the assessment time period after the processing of the movement data (e.g. visited regions) is completed. In some embodiments, score 86 is also, or alternatively, transmitted via off-board transceiver 64 to off-board device 84, where it is shared with the caregiver(s) assigned to the patient associated with patient support apparatus 20. In this manner, the caregiver is able to activate mobility assessment control 70, leave the room while the assessment is being performed, and be notified remotely when the assessment is complete, including the score 86 that was generated as a result of the assessment. As noted previously, the off-board device 84 may be a cell phone, a pager, a badge, a personal digital assistant, a nurse's station, or other electronic communication device that is adapted to share the mobility score with the caregivers associated with the patient. The communication with off-board device 84 may be direct, or it may pass through one or more intermediary devices, such as, but not limited to, the IT infrastructure necessary for automatically forwarding the assessment score 86 to a mobile communication device(s) of the appropriate personnel.

As is also shown in FIG. 6, controller 58 may also instruct display 68 to display an assessment time 88 indicating the time and/or date on which the mobility assessment was taken or completed. Still further, display 68 may include a “detailed results” control 90 that, when activated, causes display 68 to display more detailed information about the assessment. Such details may include the start and ending time of the assessment, the total amount of movement, the individual visited region count, a graphical map of the region count and/or movement sequence, one or more statistical analyses of the region count, and/or other information.

In addition to displaying the results of the assessment, controller 58 may also send a command to display 68 during the assessment time period that instructs display 68 to display a message indicating that the assessment is in progress. The message may state “Assessment in Progress,” or something similar. A light or other indicator may also or alternatively be illuminated in a manner that is indicative of the assessment being in progress. The display may also include a “pause” control that, when activated by the user, pauses the assessment and/or automatically restarts the assessment after a time period. This allows a caregiver to assist in moving the patient without the assisted movement being used in the generation of the mobility assessment score.

Alternatively, or in addition to, any of the information shown displayed in FIG. 6, control system 56 may be configured to display one or more recommended actions based upon the results of the mobility assessment. Such recommendations include, but are not limited to, any one or more of the following: (1) using a particular mattress on the support surface of patient support apparatus 20 (e.g. one designed to help reduce the interface pressures between the patient's body and the support surface); (2) turning the occupant at a particular time interval while on patient support apparatus 20; (3) inflating (or deflating) a mattress of patient support apparatus 20 to a particular level; (4) using a pressure reducing device, such as, but not limited to, a heel care boot with the patient; and/or (5) changing a dressing to, or using, a particular dressing on the patient. The display of one or more of these recommendations appears on display 68 in some embodiments. In other embodiments, the recommendations are alternatively or additionally transmitted to the same remote device(s) as the mobility assessment results are transmitted, which then displays the recommendation(s).

As was noted previously, in some embodiments of patient support apparatus 20, control system 56 performs the mobility assessment by performing other, or additional, calculations besides calculating the center of gravity of the patient. For example, in some embodiments, control system 56 performs the mobility assessment by monitoring changes in the distribution of the weight on support deck 30 due to the patient's movement, but in a way that doesn't calculate the patient's center of gravity. In such embodiments, control system 56 may be configured to perform statistical analyses on the individual outputs, or groups of outputs, from each of the load cells 54. This analysis may be similar to the analyses discussed above except that, instead of analyzing the regions that are visited, controller 58 analyzes the different values that each load cell “visits.” That is, the concept of region visits in a plane and the analysis of those visits, as was described above, is carried over to the concept of visiting numeric values in an array.

For example, suppose a load cell outputs weights from a range of zero to 200 kilograms. Such a load cell could be considered to output an array of values wherein each element in the array corresponds to one kilogram (or some other value). In such a case, controller 58 counts how many times each value is visited over the assessment time period and performs any one or more of the previously mentioned statistical analyses on those values. Thus, for example, if a particular load cell were to output a sequence of values—such as 49 kg, 46 kg, 44 kg, 45 kg, and 49 kg—during a portion of the assessment test, controller 58 would consider the load cell to have “visited” the value 49 kg twice, and visited the values 46 kg, 44 kg, and 45 kg once each. Further, the distance traveled would be calculated by summing the differences between each successive set of outputs. In this case, the distance traveled is to be ten kilograms (3 kg plus 2 kg plus 1 kg plus 4 kg). Other ways of analyzing the outputs of the load cells may also or alternatively be used.

As was also previously mentioned, in some cases mobility sensors 60 include, either exclusively or in addition to load cells 54, sensors that do not detect downward forces exerted by the patient. Such mobility sensors 60 include, but are not limited to, thermal image sensors, thermal array sensors, visual image sensors, and pressure sensing mats. When any of these types of image sensors are used, controller 58 is able to obtain more information about the movement of the individual limbs and head of the patient than is available from load cells 54. For example, with these types of sensors, controller 58 is able to measure how much the patient's arms, legs, feet, hands, head, chest, and abdomen move. With this information, controller 58 is able to determine not just the overall movement of the patient, but also the amount of movement of individual portions of the patient's body. In gathering information about the movement of different portions of the patient's body, controller 58 is programmed, in some embodiments, to apply similar statistical analyses to the individual portions of the patient's body as was described above with respect to the patient's overall body. Thus, for example, controller 58 may separately determine the sequence and number of region visits of each of the patient's right arm, left arm, right leg, left leg, trunk, and head (and/or body parts or combinations of parts). These visits are gathered during the assessment period, analyzed, and compared to a threshold amount of movement defined for each body part in order to generate an overall mobility assessment score.

In some of the embodiments where mobility sensors 60 are capable of detecting movement of specific locations of the patient's body, controller 58 is programmed to analyze the movement of one or more areas of the patient's body that are more commonly susceptible to the development of pressure ulcers, such as the back of the patient's head, sacral area, and heels. In such embodiments, controller 58 analyzes the movement of these body parts to estimate the extent to which interface pressures between these body parts and the mattress are shifted and/or offloaded during the assessment period. This analysis may be used alone to generate the assessment score, or it may be combined with the analysis of other body parts and/or the patient's entire body.

In some embodiments of patient support apparatus 20, control system 56 is adapted to not only perform a mobility assessment, but also to perform other control functions related to patient support apparatus 20. Such additional control functions may include controlling the movement of one or more components of patient support apparatus 20 and/or carrying out communication between patient support apparatus 20 and one or more off-board devices (e.g. a nurse call system, a local area network, etc.).

In some embodiments, control system 56 is adapted to also perform an exit detection function based upon, either wholly or partially, the outputs of mobility sensors 60. This exit detection function is carried out in addition to assessing the mobility of the patient based on the outputs of sensors 60. In such instances, the mobility assessment may be carried out simultaneously with the exit detection function, or it may be carried out separately. When carried out simultaneously, in addition to recording the movement of the patient for an assessment period, controller 58 also simultaneously analyzes the detected movement to determine if the patient is about to exit or not. If the patient is about to exit, controller 58 activates an alert, locally (such as via user interface 66) and/or remotely using off-board transceiver 64.

In order to carry out the exit detection function separately from the assessment control, user interface 66 is configured to include a separate control for arming and disarming the exit detection function. Thus, exit detection can be monitored at times when the mobility assessment is not being carried out or at times when both the mobility assessment and exit detection are being performed. Further, because the mobility assessment includes its own separate control 70, the mobility assessment may be carried out without arming the exit detection function. The mobility assessment function and the exit detection function can thus operate by themselves, or simultaneously with each other.

In addition to carrying out an exit detection system, control system 56 may also be adapted to carry out a scale function in which the patient's weight is measured, displayed, recorded, and, in some embodiments, transmitted to a remote device. This scale function is carried out by analyzing the outputs from one or more force sensors, such as load cells 54. This scale function, like the exit detection function, can be carried out at the same time as the mobility assessment is being performed, or it may be performed separately when no mobility assessment is being performed. Similarly, it may be carried out at the same time that either or both of the exit detection function and mobility assessment are being performed. Control system 56 can therefore, in some embodiments, measure the patient's weight while simultaneously monitoring the patient's mobility and whether the patient is about to exit or not.

Although control system 56 has been primarily described herein with respect to a patient support apparatus 20 that has been described as a bed, it will be understood that patient support apparatus 20 need not be a bed. As but one non-limiting example, patient support apparatus 20 may be implemented as a recliner. One suitable example is disclosed in commonly assigned U.S. patent application Ser. No. 62/268,549 filed Dec. 17, 2015, by inventors Anish Paul et al. and entitled PERSON SUPPORT APPARATUS WITH EXIT DETECTION SYSTEM, the complete disclosure of which is incorporated herein by reference. Other types of recliners or chairs may also incorporate control system 56 thereinto. When so incorporated, the chair or recliner includes a mobility assessment control that operates in the same manner as mobility assessment control 70 discussed above.

One modification that may be made to any of the embodiments described herein is the addition of one or more mobility sensors 60 that are worn by the patient. Such mobility sensors may include accelerometers and/or other motion sensors that detect the movement and mobility of the patient over the time period of interest. In some embodiments, the patient-worn sensors may communicate wirelessly, including times at which the patient is not present on patient support apparatus 20. Several suitable examples of such patient-worn or patient-held mobility sensors are disclosed in commonly assigned U.S. patent application Ser. No. 14/928,513 filed by inventors Richard Derenne et al. on Oct. 30, 2015, by inventors Richard Derenne et al. and entitled PERSON SUPPORT APPARATUSES WITH PATIENT MOBILITY MONITORING, the complete disclosure of which is incorporated herein by reference.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

What is claimed is:

1. A patient support apparatus comprising:
a support surface adapted to support thereon an occupant of the patient support apparatus;
a sensor adapted to detect movement of the occupant while the occupant is supported on the support surface;
a user interface having a mobility assessment control; and
a controller adapted to monitor outputs from the sensor in response to the mobility assessment control being activated, the controller further adapted to generate a mobility score based on the outputs from the sensor over a predetermined time period.

2. The patient support apparatus of claim 1 wherein the controller is further adapted to display the mobility score on the user interface.

3. The patient support apparatus of claim 1 wherein the sensor comprises a plurality of force sensors adapted to detect downward forces exerted by the occupant on the support surface and the controller is adapted to use the outputs from the force sensors to determine a distribution of the occupant's weight on the support surface and to detect changes in distribution of the occupant's weight.

4. The patient support apparatus of claim 3 wherein the controller records both a number of changes in the distribution of the occupant's weight occurs over the predetermined time period and a magnitude of the changes in the distribution of the occupant's weight, the controller further adapted to use both the number and magnitude of the changes in the distribution the occupant's weight when generating the mobility score.

5. The patient support apparatus of claim 1 further comprising a transceiver adapted to transmit the mobility score to a device off-board the patient support apparatus.

6. The patient support apparatus of claim 1 wherein the controller is further adapted to recommend an action based on a value of the mobility score, and the action is designed to reduce a possibility of the occupant developing pressure ulcers while supported on the support surface.

7. The patient support apparatus of claim 6 wherein the action comprises recommending one or more of the following: (1) using a particular mattress on the support surface of the patient support apparatus; (2) turning the occupant at a particular time interval while on the support surface of the patient support apparatus; (3) inflating a mattress on the support surface to a particular level; and (4) using a pressure reducing device with the occupant while on the support surface.

8. The patient support apparatus of claim 1 wherein the controller is further adapted to:
define an area over which the occupant may move while supported on the support surface;
divide the area into a plurality of regions;
determine which region the occupant's location on the support surface corresponds to; and
generate a record of the regions which the occupant's location has corresponded to during the predetermined time period.

9. The patient support apparatus of claim 8 wherein the controller is further adapted to analyze the record to determine how many times the occupant's location has visited each region during the predetermined time period.

10. The patient support apparatus of claim 9 wherein the controller is further adapted to determine at least one of the following: (1) a concentration value of the visited regions; (2) a distribution value of the visited regions; (3) how many times the occupant has moved more than a threshold number of regions; and (4) how many times the occupant has moved to the same region.

11. The patient support apparatus of claim 1 wherein the controller is adapted to automatically monitor the outputs from the sensor and generate the mobility score in response to at least one of the following: a changing of a work shift for a nurse assigned to the occupant; a passage of a predetermined amount of time; an assignment of a new occupant to the patient support apparatus; and an occurrence of an event.

12. The patient support apparatus of claim 1 wherein the controller is further adapted to automatically forward the mobility score to a caregiver communication device after the predetermined time period has passed.

13. A patient support apparatus comprising:
a support surface adapted to support thereon an occupant of the patient support apparatus;
a sensor adapted to detect movement of the occupant while the occupant is supported on the support surface, the movement being detected over an area subdivided into regions; and
a controller adapted to use outputs from the sensor to determine which region of the area the occupant's location on the support surface corresponds to, and to generate a record of the regions which the occupant's location has corresponded to over a time period.

14. The patient support apparatus of claim 13 wherein the controller is further adapted to analyze the record to determine how many times the occupant's location has visited each region over the time period.

15. The patient support apparatus of claim 14 wherein the controller is further adapted to determine at least one of the following: (1) a concentration value of the visited regions; (2) a distribution value of the visited regions; (3) how many times the occupant has moved more than a threshold number of regions; and (4) how many times the occupant has moved to the same region.

16. The patient support apparatus of claim 14 wherein the controller is further adapted to determine one or more clusters of the visited regions and to display the clusters.

17. The patient support apparatus of claim 13 wherein the controller is further adapted to generate a mobility score for the occupant based on the outputs from the sensor taken over the time period, the mobility score being automatically generated in response to at least one of the following: a mobility assessment control being activated; a changing of a work shift for a nurse assigned to the occupant; a passage of a predetermined amount of time; and an assignment of a new occupant to the patient support apparatus.

18. The patient support apparatus of claim 17 wherein the controller is further adapted to determine if the occupant exits the patient support apparatus during the time period and to use data regarding the occupant's exit when generating the mobility score.

19. The patient support apparatus of claim 17 wherein the sensor comprise at least one of the following: a video camera, a plurality of force sensors adapted to detect downward forces exerted by the occupant on the support surface; a thermal image sensor; and an accelerometer adapted to detect accelerations of a support deck on which the support surface is located.

20. The patient support apparatus of claim 19 wherein the controller is adapted to use the outputs from the sensor to determine a distribution of the occupant's weight on the support surface and to detect changes in distribution of the occupant's weight.

21. The patient support apparatus of claim 20 wherein the controller records both a number of changes in the distribution of the occupant's weight occurs over the time period and a magnitude of the changes in the distribution of the occupant's weight, the controller further adapted to use both the number and magnitude of the changes in the distribution the occupant's weight when generating the mobility score.

22. The patient support apparatus of claim 17 wherein the controller is further adapted to recommend at least one of the following actions if the mobility score meets a predetermined threshold:
(1) using a particular mattress on the support surface of the patient support apparatus; (2) turning the occupant at a particular time interval while on the support surface of the patient support apparatus; (3) inflating a mattress on the support surface to a particular level; and (4) using a pressure reducing device with the occupant while on the support surface.
